(19) United States
(12) Patent Application Publication
(10) Pub. No.: US 2019/0358263 A1
(43) Pub. Date: Nov. 28, 2019

(54) Compositions and Methods for Cell Therapy

(71) Applicant: Oslo Universitetssykehus HF, Oslo (NO)

(72) Inventors: Sebastien Walchli, Oslo (NO); Else Marit Inderberg, Oslo (NO); Gunnar Kvalheim, Oslo (NO); Gustav Gaudernack, Sandvika (NO); Nadia Mensali, Oslo (NO)

(21) Appl. No.: 16/467,400

(22) PCT Filed: Dec. 7, 2017
(86) PCT No.: PCT/EP2017/081880

§ 371 Date: Jun. 6, 2019

Related U.S. Application Data
(60) Provisional application No. 62/431,168, filed on Dec. 7, 2016.

(51) Int. Cl. A61K 35/17; C07K 14/705; C12N 5/0783

(52) U.S. Cl. CPC A61K 35/17; C12N 5/0636; C12N 5/0646; C07K 14/70521

(57) ABSTRACT

The present invention relates to cancer immunotherapy. In particular, provided herein are compositions and methods for improving the efficacy of cell therapies cancer and other diseases.

### FIELD OF INVENTION

The present invention relates to cancer immunotherapy. In particular, provided herein are PD-1 molecules and immune effector cells expressing the molecule, as well as compositions and methods for improving the efficacy of T-cell or natural killer cell (NK cell) therapies in cancer and other diseases.

### BACKGROUND OF THE INVENTION

Despite advances in our understanding and management of cancer, the majority of cancer patients will still die of the disease. For many patient groups, prognosis is dire, with few or no available curative treatment regimens. Furthermore, resistance development and disease relapse is commonplace. There is therefore an unmet need for the development of novel therapeutics founded on fundamentally different mechanisms of action.

The ability of the immune system to fight cancer has been clearly demonstrated in recent years, highlighted by the massive clinical and commercial success of the immune checkpoint inhibitors. One of the key advantages offered by these drugs is that they have the potential to treat several cancer types and are supplied in an off-the-shelf manner. In contrast to personalized immunotherapies such as adoptive T-cell therapy or dendritic cell (DC) vaccines, they do not rely on customization for each patient. On the downside, only 10-30% of patients respond adequately, and checkpoint inhibitors have a challenging safety profile.

PD-1 is a receptor expressed in the cell membrane of a variety of immune effector cells such as activated T-cells, B-cells, macrophages and natural killer cells. PD-1 is an abbreviation for “Programmed cell death protein 1”, also known as CD279 (cluster of differentiation 279). Upon binding of its ligand, PDL-1, it may deliver a signal into immune effector cells inhibiting proliferation, cytokine production and/or cytolytic function. In particular, it is assumed to bring T-cells to anergy.

While not being limited to a particular mechanism, it is contemplated that PD-1 signaling may be partly responsible for the low clinical efficacy of many therapeutic immune effector cells when PDL-1 is expressed on target cells (often the case of tumors). The exact signaling mechanism of PD-1 is not yet fully understood. However, the wild type PD-1 intracellular domain comprises an immunoreceptor tyrosine-based inhibition motif, ITIM. In T-cells, it is suggested that upon binding to PDL-1, PD-1 receptors may cluster in the cell membrane, ITIM may get phosphorylated and trigger an inhibitory signaling cascade, i.e. a PD-1 suppression signal. This suppression signal may counteract T-cell receptor (TCR) stimulation and activation of T-cells.

It is known that blocking PD-1/PDL-1 axes may prevent inhibition of immune effector cells. Liu et al, Cancer Res; 76(6) Mar. 15, 2016 disclosed that treatment of mice bearing solid tumors with T-cells expressing PD-1 molecules comprising a CD28 signaling domain in addition to a chimeric antigen receptor (CAR) led to significant regression in tumor volume. On the other hand, it was also mentioned that T-cells expressing a tailless PD-1 molecule in addition to a chimeric antigen receptor demonstrated significantly worse tumor control compared to T-cells comprising the chimeric antigen receptor alone.

US20160256488 mentions a construct “P2:CD19BBZETA-ires-Truncated PD-1” wherein the PDL-1 inhibition ratio was higher than for the comparable construct comprising the wild-type PD-1 receptor.

Accordingly, there is a need for alternative therapies targeting PDL-1-expressing cells.

### SUMMARY OF THE INVENTION

The present invention relates to cancer immunotherapy. In particular, provided herein are PD-1 molecules and immune effector cells expressing the molecule, as well as compositions and methods for improving the efficacy of T-cell or NK cell therapies in cancer and other diseases.

In a first embodiment, the present disclosure provides a PD-1 molecule comprising an extracellular domain, a transmembrane domain and an intracellular domain, wherein the intracellular domain comprises a juxtamembrane domain and wherein the intracellular domain is lacking other signaling domains.

It is found that immune effector cells expressing in their cell membrane a truncated PD-1 molecule comprising a juxtamembrane domain have several advantages. Such PD-1 molecules may be generated by truncation of the signaling domain, in particular the truncation of the C-terminal sequence comprising the immunoreceptor tyrosine-based inhibition motif (ITIM) represented by the amino acid sequence as depicted in SEQ ID NO 5 (VDYGEL) and the immunoreceptor tyrosine-based switch motif (ITSM) represented by the amino acid sequence as depicted in SEQ ID NO 6 (TEYATI).

Accordingly, in some aspects of the first embodiments, it is provided a PD-1 molecule comprising an extracellular domain, a transmembrane domain and an intracellular domain, wherein the intracellular domain comprises a juxtamembrane domain and wherein the intracellular domain does not comprise an amino acid sequence corresponding to SEQ ID NO 5 and/or SEQ ID NO 6. In another aspect of this embodiment, the intracellular domain is lacking the ITIM motif and/or ITSM motif.

In one aspect of the first embodiment, the juxtamembrane domain comprises a amino acid sequence corresponding to SEQ ID NO 7 (AARGTIGARRTGQPLKEDPSAVP) or SEQ ID NO 8 (CSRAARGTIGARRTGQPLKEDPSAVPVFS).

In a second aspect of the first embodiment, the intracellular domain comprises a amino acid sequence corresponding to SEQ ID NO 8 (CSRAARGTIGARRTGQPLKEDPSAVPVFS).

In a third aspect of the first embodiment, the juxtamembrane domain consists of the amino acid sequence corresponding to SEQ ID NO 9 (ICSRAARGTIGARRTGQPLKEDPSAVPVFS)

In a second embodiment, the present disclosure provides an immune effector cell expressing a PD-1 molecule according to the first embodiment in its cell membrane.

It is also provided a PD-1 molecule as described, an immune effector cell expressing said PD-1 molecule and compositions for use in medicine, such as in therapy. In particular, it is provided PD-1 molecules, cells expressing said PD-1 molecule and compositions for use in immunotherapy, such as for generating an immune response in a patient.

Accordingly, in a third embodiment, a PD-1 molecule according to the first embodiment or an immune effector cell according to the second embodiment or a composition is provided for use in medicine or in therapy, such as in immunotherapy, or to generate an immune response in a patient. In different aspects of this embodiment, the PD-molecule or immune effector cell or composition may be administered with an immune system targeting molecule to generate an antigen-specific immune response.

In further embodiments, provided herein is a method of generating an antigen-specific immune response in patients, comprising:

infusion to the patients of T-cells or NK cells expressing 1) a high affinity truncated PD-1 comprising the extracellular domain, transmembrane domain, and juxtamembrane domain and lacking the signaling domain and 2) an immune system targeting molecule.

In some embodiments, the PD-1 comprises an amino acid sequence selected from, for example, SEQ ID NO:2 or sequences that are at least 90% (e.g., at least 90%, 91%, 92%, 93%, 94%, 95%, 96%, 97%, 98%, or 99%) identical to SEQ ID NO:2. In some embodiments, the PD-1 comprises a mutation that increases affinity for a binding partner (PDL-1). In some embodiments, the mutation is an A132L, an A132I or an A132V mutation, wherein the position 132 is as defined in SEQ ID NO 1. In some embodiments, the mutation is an A112L, an A112I or an A112V mutation, wherein the position 112 is as defined in SEQ ID NO 4. In some embodiments, a nucleic acid encoding the PD-1 is in a vector, or is not in a vector. In some embodiments, the vector is a plasmid or a self-inactivating vector (e.g., retroviral vector). In some embodiments, the immune system targeting molecule is a chimeric antigen receptor or T-cell receptor, although the present invention is not limited to a particular immune system targeting molecule. In some embodiments, the antigen is a cancer antigen. In some embodiments, the administration treats cancer in said subject. In some embodiments, NK cells (e.g., NK-92 cells) are engineered to express CD3 (e.g., via a nucleic acid encoding CD3 as described in WO2016116601).

Further embodiments provide an in vitro or ex vivo T-cell or NK-cell, comprising: 1) a high affinity truncated PD-1 comprising the extracellular domain, transmembrane domain, and juxtamembrane domain and lacking the signaling domain and 2) an immune system targeting molecule.

Yet other embodiments provide the use of such T-cells or NK-cells to generate an antigen specific immune response in a subject.

Additional embodiments are described herein.

### DESCRIPTION OF THE DRAWINGS

FIG. 1 shows the frequency (%) of NK-92 cells that are activated upon 5 h of co-culture with target cells (1:2 Effector:Target ratio) that are either PDL-1 negative or positive. NK-92 cells were mRNA electroporated to express either full length PD-1 (flPD-1), (mRNA encoding SEQ ID NO:1), or truncated PD-1 (tPD-1), (mRNA encoding SEQ ID NO:2). Target cells used in the experiments are: EBV-LCL, K562 A2-, K562 A2+. Target cells were mRNA electroporared to express PDL-1. In the case of EBV-LCL it is important to remind that these cells express endogenous PDL-1. Activation of NK-92 cells was measured as CD107a mobilization by flow cytometry-based assay. NK-92 cells were identified by surface expression of the lineage marker CD56. Activated NK-92 cells are identified as CD56+/CD107a+.

FIG. 2 shows the killing ability of NK-92 cells expressed as percent (%) of target lysis. NK-92 cells were mRNA electroporated to express either full length PD-1, flPD-1 (mRNA encoding SEQ ID NO:1), or truncated PD-1, tPD-1 (mRNA encoding SEQ ID NO:2). K562 A2—were used as target cells; K562-A2—were either mRNA electroporated to express PDL-1 or mock electroporated. NK-92 and K562 A2—were co-cultured for two hours at 25:1 Effector:Target ratio. Europium assay for performed to measure the target lysis (see experimental session).

FIG. 3a shows the frequency (%) of UK-92-TCR cells that are antigen specifically activated. UK-92-TCR expressing Rad1 TCR (TCR specific for TGFbRII derived peptide 621, p621) were mRNA electroporated to express either full length PD-1, flPD-1 (mRNA encoding SEQ ID NO:1) or truncated PD-1, tPD-1 (mRNA encoding SEQ ID NO:2). Target cells (K562 A2+) were either loaded or not with the specific peptide (p621) and they were also mRNA electroporated to express PDL-1 or mock electroporated. UK-92-TCR and target cells were co-cultured 5 h at 1:2 Effector:Target ratio and CD107a mobilization assay was performed to measure antigen specific UK-92-TCR activation. Activated UK-92-TCR cells were identified as CD56+/CD107+ cells.

FIG. 3b shows the frequency (%) of UK-92-TCR cells that are antigen specifically activated. UK-92-TCR expressing DMFS TCR (TCR specific for Mart-1 peptide) were mRNA electroporated to express either full length PD-1, flPD-1 (mRNA encoding SEQ ID NO:1) or truncated PD-1, tPD-1 (mRNA encoding SEQ ID NO:2). The experimental setting is the same presented in FIG. 3a.

FIG. 4 shows the frequency (%) of antigen specific activation of CD8+ effector T-cells. T-cells were mRNA electroporated to express Rad-1 TCR alone or in combination with either flPD-1 (mRNA encoding SEQ ID NO:1) or tPD-1 (mRNA encoding SEQ ID NO:2). T-cells mock for both Rad1 TCR and PD-1 were also used as a control. Target cells (K562 A2+) were either loaded with the specific peptide, p621, (p621 is recognized by Rad1 TCR) or with an irrelevant peptide, Mart1. Target cells were also mRNA electroporated to express PDL-1 or mock electroporated. T cells and target cells were co-cultured 5 h at 1:2 Effector:Target ratio and CD107a mobilization assay was performed to measure antigen specific T-cell activation. Antigen specific T-cell activation was measured as CD8+/CD107+ T-cells.

FIG. 5a shows the frequency (%) of antigen specific activation of CD4+ helper T-cells. T-cells (bulk of both CD8+ Cytotoxic T-cells and CD4+ Helper T-cells) were mRNA electroporated to express Rad-1 TCR alone or in combination with either flPD-1 (mRNA encoding SEQ ID NO:1) or tPD-1 (mRNA encoding SEQ ID NO:2). Target cells (K562 A2+) were loaded with the specific peptide, p621, and they were either mRNA electroporated to express PDL-1 or mock electroporated. T cells and target cells were co-cultured 5 h at 1:2 Effector:Target ratio and TNFalpha production was measured by intracellular cytokine staining assay. Antigen specific T-cell activation was measured as CD4+/TNFalpha+ T-cells

FIG. 5b shows the fraction of CD8+ Cytotoxic T-cells that are activated and produce TNFalpha upon encounter with the target cells presenting the cognate antigen. Data shown in FIG. 5b come from the experiment described in FIG. 5a.

FIG. 6 shows T-cell cytoxicity. The lytic capacity of T-cells was measured by BL1 assay (see experimental session) and it was measured as % of lysis of tumor cells overtime. T cells were mRNA electroporated to express Rad-1 TCR alone or in combination with either flPD-1 (mRNA encoding SEQ ID NO:1) or tPD-1 (mRNA encoding SEQ ID NO:2). The colon-rectal cancer cell line HCT116 was used as target cells. HCT 116 cells express endogenous TgFbRII antigen (the cognate antigen of Rad1 TCR). HCT116 used in the killing assay were either retrovirally transduced to express PDL-1 or mock transduced. T-cells and target cells were co-cultured at 25:1 Effector:Target ratio and killing was measured overtime by BLI assay.

FIG. 7 shows T-cell cytoxicity. Similar to FIG. 6 the lytic capacity of T-cells was measured by BL1 assay (see experimental session) and it was measured as % of lysis of target cells overtime. In this experiment T-cells were retrovirally transduced to stably express the Rad1 TCR, alone, or in combination with either the flPD-1 (construct encoding SEQ ID NO:1) or the tPD-1 (construct encoding SEQ ID NO:2). The Granta cell line was used as target cells. Target cells were loaded with the specific peptide (p621) and they were mRNA electroporated to express PDL-1. T-cells and target cells were co-cultured at 25:1 effector:target ratio and killing was measured overtime by BLI assay.

FIG. 8 shows expression of PD-1 on T-cells after mRNA electroporation. Expression of PD-1 is detected in T-cells by surface staining of the cells with antibody specific for PD-1 receptor.

FIG. 9 shows expression of PDL-1 in target cells after electroporation. Expression of PDL-1 is detected in target cells (K562) by surface staining of the cells with antibody specific for PDL-1 molecule.

FIG. 10 shows titration of the mRNA encoding for PDL-1 gene. Expression of PDL-1 is detected in Mino cells by surface staining of the cells with antibody specific for PDL-1 molecule

FIG. 11a,b show the design of the construct for clinical use: The TCR coding sequence comprising TCRa and TCRb linked through a 2A ribosome skipping sequence is modified by STOP codon removal and followed by an additional 2A sequence which is placed upstream of the tPD-1 coding sequence. This module is subcloned between LTR sequence of a retroviral construct. Therefore, the TCR transcription is close to equimolarity with tPD-1.

FIG. 12 shows the amino acid sequence representing a wild type full length PD-1 molecule (flPD-1, SEQ ID NO:1) comprising an N-terminal signal peptide, an extracellular domain, a transmembrane domain and an intracellular domain, wherein the (ITIM) represented by the amino acid sequence VDYGEL and the immunoreceptor tyrosine-based switch motif (ITSM) represented by the amino acid sequence TEYATI are boxed. The juxtamembrane domain is outlined in bold font. The N-terminal signal peptide (underlined) is believed to be trimmed off during maturation, thus the mature receptor may not comprise this sequence.

FIG. 13 shows the amino acid sequence representing a truncated PD-1 molecule (tPD-1, SEQ ID NO:2). The PD-1 molecule comprises an N-terminal signal peptide, an extracellular domain, a transmembrane domain and an intracellular domain, wherein the intracellular domain comprises a juxtamembrane domain and wherein the intracellular domain is lacking other signaling domains. The juxtamembrane domain is outlined in bold font. The N-terminial signal peptide (underlined) is believed to be trimmed off during maturation, thus the mature receptor may not comprise this sequence.

FIG. 14 shows the amino acid sequence representing a mature truncated PD-1 molecule (tPD-1, SEQ ID NO:3). The PD-1 molecule comprises an extracellular domain, a transmembrane domain and an intracellular domain, wherein the intracellular domain comprises a juxtamembrane domain and wherein the intracellular domain is lacking other signaling domains. The juxtamembrane domain is outlined in bold font.

FIG. 15 shows the amino acid sequence representing a mature truncated PD-1 molecule (tPD-1 variant, SEQ ID NO:4). The PD-1 molecule comprises an extracellular domain, a transmembrane domain and an intracellular domain, wherein the intracellular domain comprises a juxtamembrane domain and wherein the intracellular domain is lacking other signaling domains. The juxtamembrane domain is outlined in bold font. The underlined position comprises a leucine residue. In the wild type sequence, this position comprises an alanine residue. The substitution of alanine with leucine in this position is referred to as A112L.

### DEFINITIONS

As used herein, the term “PD-1 molecule” means receptors able to bind PDL-1 (Programmed death-ligand 1) under physiological conditions.

As used herein, the term “PDL-1” means the natural ligand for the wild type PD-1 molecule. This ligand is also known as cluster of differentiation 274 (CD274) or B7 homolog 1 (B7-H1).

As used herein, “wild type PD-1 molecules” are naturally occurring receptors able to deliver an inhibitory signal into immune effector cells upon binding to PDL-1 under physiological conditions.

As used herein, the term “truncated PD-1 molecule” means receptors able to bind PDL-1 under physiological conditions, but lacking the ability to deliver an inhibitory signal into immune effector cells upon binding to PDL-1 under physiological conditions. Such receptors are abbreviated tPD-1 or tPD1 herein.

As used herein, “high affinity truncated PD-1”, means receptors able to bind PDL-1 under physiological conditions with the same or increased affinity compared to the wild type PD-1 receptor, but lacks the ability to deliver an inhibitory signal into immune effector cells upon binding to PDL-1 under physiological conditions.

As used herein, “physiological conditions” means in vivo conditions, or in vitro at 37° C. in a suitable medium.

As used herein, “A132L”, means that alanine in position 132 in an amino acid sequence is substituted with a Leucine residue.

As used herein, “extracellular domain”, means the part of the receptor facing the extracellular environment when expressed in the cell membrane of an immune effector cell. The extracellular domain comprises a structure able to bind PDL-1 under physiological conditions.

As used herein, “transmembrane domain”, means the part of the receptor which tend to be embedded in the cell membrane when expressed by an immune effector cell.

As used herein, “juxtamembrane domain”, means an intracellular part of the wild type PD-1 receptor located between the transmembrane domain and the ITIM motif in the wild type PD-1 molecule.

As used herein, “immune effector cell”, means mature lymphocytes suitable for therapy, including cytotoxic T-cells, helper T-cells and natural killer cells.

### DETAILED DESCRIPTION OF THE INVENTION

The present invention relates to cancer immunotherapy. In particular, provided herein are a PD-1 molecule and an immune effector cell expressing the molecule, as well as compositions and methods for improving the efficacy of T-cell or NK-cell therapies in cancer and other diseases.

When truncated PD-1 molecules comprising a juxtamembrane domain are expressed in the cell membrane of an immune effector cell, they may allow the immune effector cell to bind to PDL-1 expressing cells without delivering the PD-1 suppression signal into the immune effector cell. tPD-1 can have a suppressive effect by two mechanisms: 1) it competes out the endogenous PD-1, “chasing” it from the immune synapse, therefore removing the negative signaling by avoiding PDL-1 binding and PD-1 clustering which lead to inhibitory signal; and/or 2) by reinforcing the binding of the two cells through PD-1/PDL-1 contact, it increases the probability of the formation of an active immune synapse, leading to an improved effector cell stimulation.

When such immune effector cells also express receptors, i.e. Chimeric Antigen Receptors (CAR) or T-cell Receptors (TCR), with specificity for tumor associated antigens in their cell membrane, the immune effector cells may be used for anti-tumor purposes with several advantages as disclosed herein.

In contrast to the prior art truncated PD-1 molecules, the truncated PD-1 molecules herein can deliver an unknown signal or a more moderate signal into immune effector cells which may be beneficial in a clinical setting. Since the ITIM domain is ablated, the truncated PD-1 molecules can further avoid delivery of negative signals. The intracellular domain from the transmembrane domain to the ITIM may be conserved in order to not modify the membrane localization of the product. Accordingly, based on the experiments herein, it is suggested that the juxtamembrane domain may comprise critical binding sites for scaffolding or structural proteins. Accordingly, the correct targeting of tPD-1 is likely to further facilitate a proper improving effect.

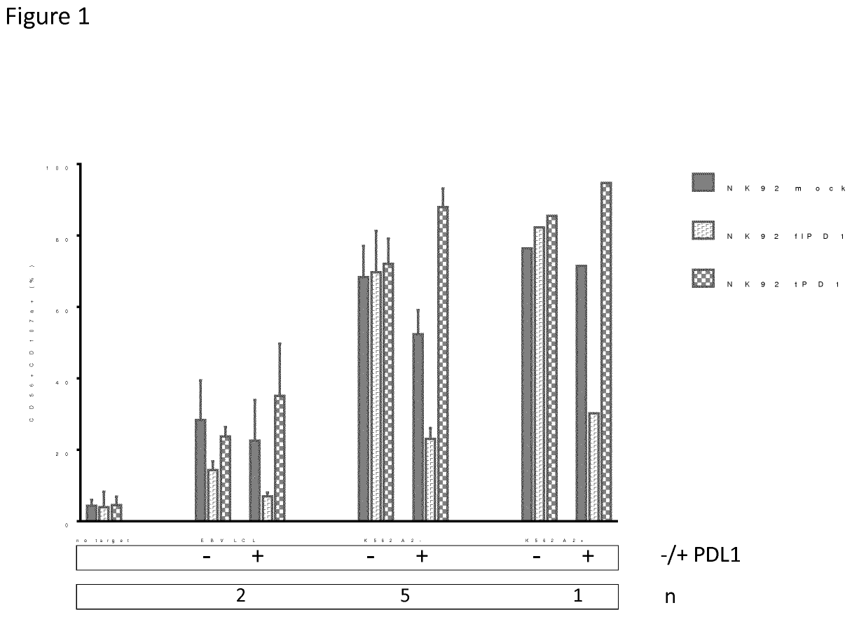

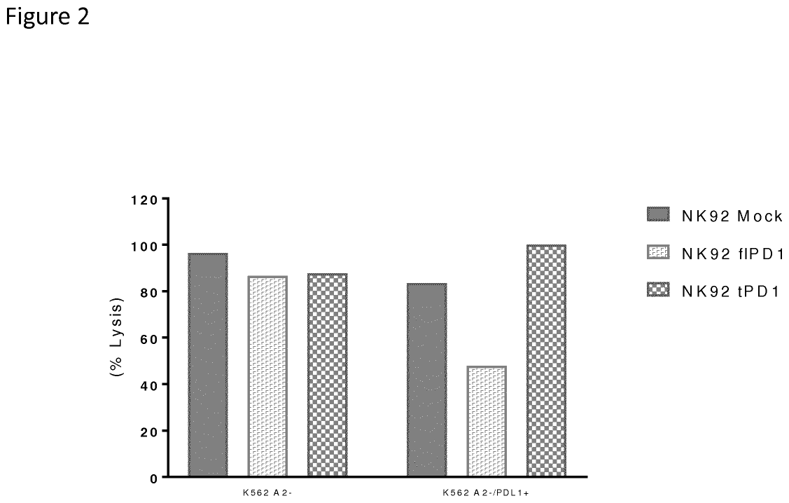

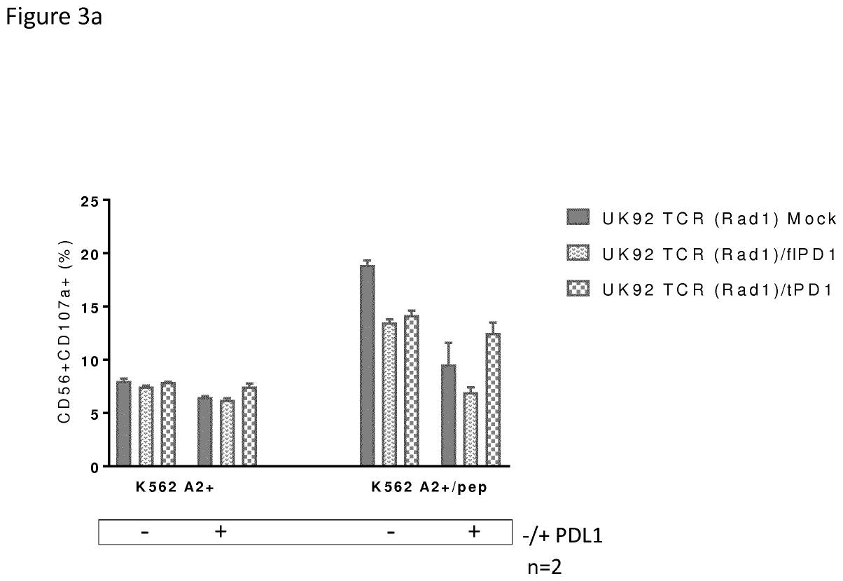

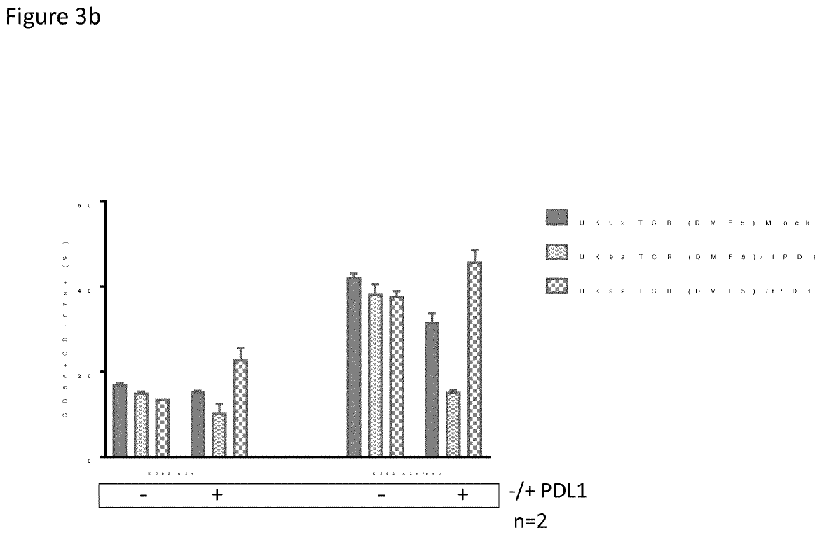

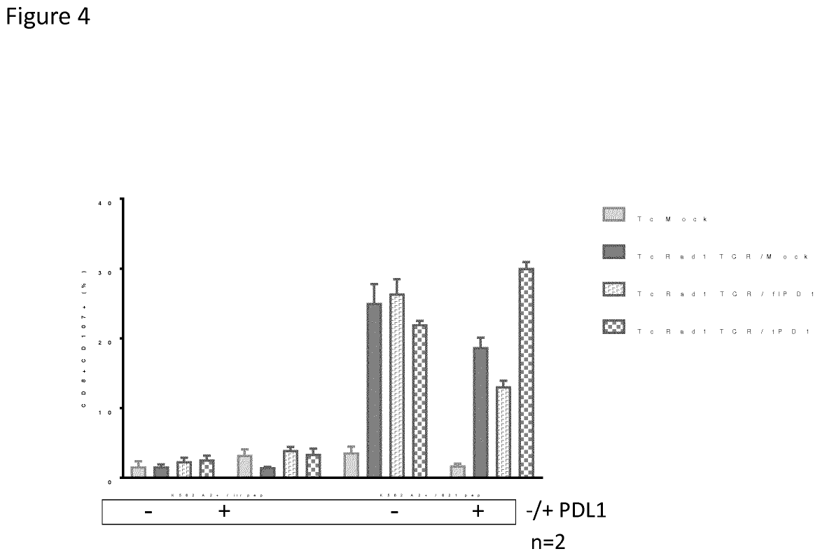

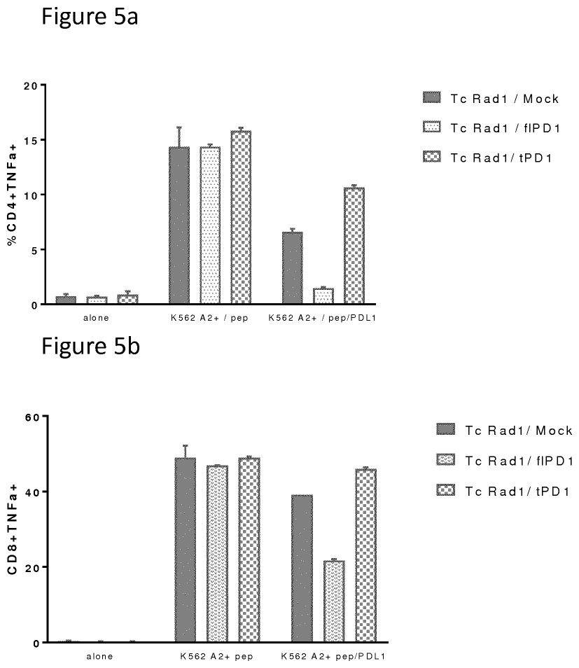

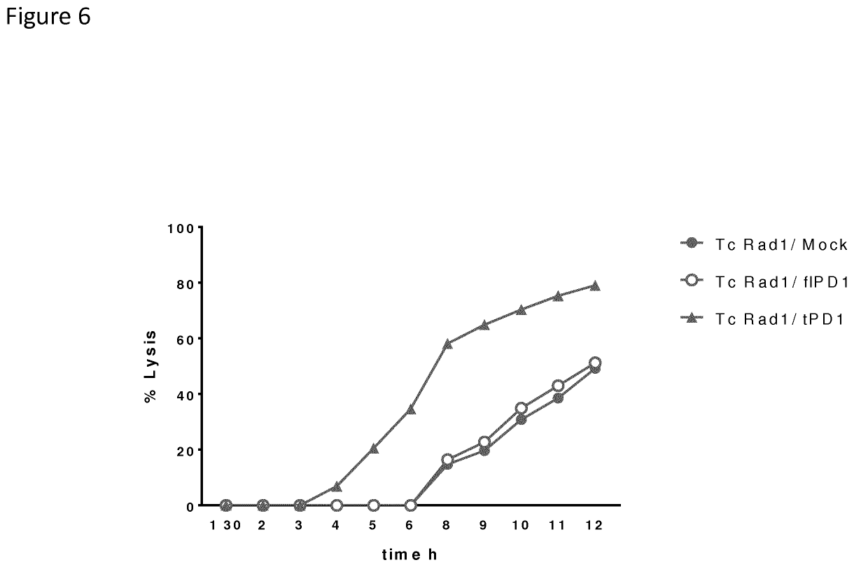

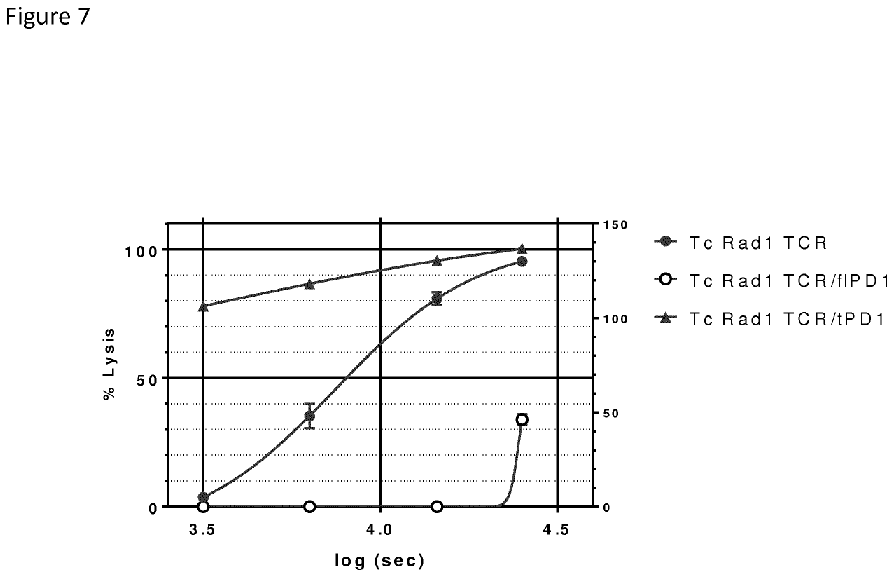

As shown in FIG. 1, FIG. 3a, FIG. 3b and FIG. 4 the expression of the truncated PD-1 molecule comprising a juxtamembrane domain lead, in fact, to an increased fraction of CD107a positive cells. As demonstrated in example 2 and visualized in FIG. 2, the cytotoxicity of a natural killer cell line (NK-92) was increased by expression of the truncated PD-1 molecules comprising the juxtamembrane domain when the target cells were PDL-1 positive. It can also be seen from FIG. 2 that the truncated PD-1 molecule comprising the juxtamembrane domain did not affect the specificity of the immune effector cells, since in the absence of PDL-1, no difference was observed between the effector cells mock transfected or transfected with full length (fl) or truncated (t) PD-1. As demonstrated in example 7 and visualized in FIG. 7, the cytotoxicity of T-cells was increased by expression of the truncated PD-1 molecules comprising the juxtamembrane domain when the target cells were PDL-1 positive. Such improved killing kinetics is usually desired for adoptive cell therapy relying on cells unable to proliferate. It also validates the in vitro efficacy of the construct, because if the targeting was wrong, or if the juxtamembrane domain was also participating to negative signal it would be expected to have a negative effect as shown by US20160256488 and Liu et al, Cancer Res; 76(6) Mar. 15, 2016. Without being bound by theory, it is suggested that the difference between the aforementioned prior art and the present invention is due to the design of a construct comprising the juxtamembrane domain. Similar results are reported in example 6 and showed in FIG. 6. Furthermore, as shown in FIG. 5a and FIG. 5b, expression of the truncated PD-1 molecule comprising the juxtamembrane domain also lead to increased fraction of TNF-α positive T-cells. This is complementing the observation of degranulation (CD107a) and killing, it suggests that the construct is not modifying the positive signals from the TCR. Indeed, although the killing is conserved, one could expect that the cytokine release could still be inhibited by the signaling which could come from the juxtamembrane domain. We here show that this is not the case.

Without being bound by theory, the advantages conferred by the truncated PD-1 molecules comprising a juxtamembrane domain may be related to less exhaustion of the immune effector cells expressing them compared to truncated PD-1 molecules comprising a CD28 signaling domain. In addition, it has been shown that CAR construct expressing a CD28 signaling domain do not lead to a therapeutically favorable metabolic pathway (PMID: 26885860), thus PD-1-CD28 fusion might lead to the same phenotype. The truncated PD-1 is neutral, and according to our data does not modify the TCR derived stimulation. In addition, the extracellular domain being similar to the endogenous PD-1, its binding to target cells expressing PDL-1 might even improve the binding and the avidity for the target when expressed in immune effector cells.

Accordingly, provided herein are compositions and methods suitable for use in adoptive cell therapy. In some embodiments, the present disclosure provides a therapeutic transgene (e.g., CAR, TCR, etc.) in combination with a truncated PD-1 unable to signal (e.g., comprising the extracellular domain (ECD), the transmembrane domain and the juxtamembrane domain, but lacking the ITIM motif). The advantages of such embodiments include the following:

1) the T-cell carries the “blocking construct” together with the therapeutic construct;

2) no PD-1 signaling that interferes with TCR signaling;

3) the ECD competes with endogenous PD-1 for PDL-1 binding;

4) the binding of ECD with PDL-1 increases the binding between the effector cell and the target, which will favor the killing of cancer cells; and

5) if the cancer is PDL-1 negative, the construct does not interfere with recognition (see FIG. 1).

In some embodiments, a substitution of an alanine residue in the wild type PD-1 sequence with a Leucine residue (A132L; WO2014124217; herein incorporated by reference in its entirety) improves the affinity of the PD-1 for PDL-1.

In some embodiments, the truncated PD-1 construct is provided as a transgene. In mRNA-based therapy embodiments, truncated PD-1 construct may be co-electroporated with the therapeutic construct. In some embodiments utilizing virus-based transduction, truncated PD-1 is connected to the therapeutic construct with a ribosome skipping sequence (2A).

Accordingly, in some embodiments, provided herein are expression constructs for expression of truncated PD-1 molecule. The present invention is not limited to particular PD-1 sequences. Any PD-1 that lacks signaling activity is specifically contemplated. In some embodiments, the PD-1 comprises the extracellular domain (ECD), the transmembrane domain and the juxtamembrane domain, but lacks the ITIM motif. In some embodiments, the amino acid sequence of the truncated PD-1 is selected from SEQ ID NO:2 and sequences that are at least 90% (e.g., at least 90%, 91%, 92%, 93%, 94%, 95%, 96%, 97%, 98%, or 99%) identical to SEQ ID NO:2. In some embodiments, PD-1 comprises one or more additional amino acid changes (e.g., A132L) and/or additional conservative or non-conservative changes. In some embodiments, PD-1 is engineered to increase the affinity towards PDL-1.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acids substitution groups are: valine-leucine-isoleucine, phenylalanine-tyrosine, lysine-arginine, alanine-valine, and asparagine-glutamine.

Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic hydroxyl; (4) aromatic (phenylalanine, tyrosine, tryptophan); (5) amide (asparagine, glutamine); and (6) sulfur containing (cysteine and methionine) (e.g., Stryer ed., Biochemistry, pg. 17-21, 2nd ed., WH Freeman and Co., 1981).

In some embodiments, a variant includes “non-conservative” changes (e.g., replacement of a glycine with a tryptophan). Guidance in determining which amino acid residues can be substituted, inserted, or deleted without abolishing biological activity can be found using computer programs.

In some embodiments, PD-1 is provided to a cell as a gene expression construct (e.g., vector).

The present invention is not limited to a particular expression vector. In some embodiments, vectors are self-inactivating. In some embodiments, vectors are retroviral vectors (e.g., lentiviral vectors). Table 1 provides a summary of exemplary suitable vectors.

TABLE 1

| Virus | Advantages | Disadvantages |
|---|---|---|
| Adenovirus | High titer | Immunogenic |
| | High gene expression | Does not integrate into genome |
| | Can infect non-dividing cells | |
| | Accepts very large cassettes (40 kb) | |
| Adeno-associated virus | Can infect non-dividing cells | Accepts small cassettes (4 kb) |
| | Relatively safe in humans | Low transduction efficiency in |
| | | hematopoietic cells |
| Alphavirus (Sindbis) | Can infect non-dividing cells | Toxic to cells |
| | High titer | Does not integrate into genome |
| | High transduction efficiency | |
| | High gene expression | |
| Lentivirus | Stably incorporated into genome | New to field |
| | Can infect non-dividing cells | Safety uncertain in humans |
| Retrovirus | Stably incorporated into genome | Infects only dividing cells |
| | Relatively safe in humans | |
| | High titer | |
| | Accepts large cassettes (8 kb) | |

The present invention is not limited to retroviral vectors. Large numbers of suitable vectors are known to those of skill in the art, and are commercially available. Such vectors include, but are not limited to, the following vectors: 1) Bacterial—pQE70, pQE60, pQE 9 (Qiagen), pBS, pD10, phagescript, psiX174, pbluescript SK, pBSKS, pNH8A, pNH16a, pNH18A, pNH46A (Stratagene); ptrc99a, pKK223 3, pKK233 3, pDR540, pRIT5 (Pharmacia); and 2) Eukaryotic—pWLNEO, pSV2CAT, pOG44, PXT1, pSG (Stratagene) pSVK3, pBPV, pMSG, pSVL (Pharmacia). Any other plasmid or vector may be used as long as they are replicable and viable in the host. In some embodiments of the present invention, mammalian expression vectors comprise, along with an expression cassette as described herein, an origin of replication, any necessary ribosome binding sites, polyadenylation sites, splice donor and acceptor sites, transcriptional termination sequences, and 5′ flanking non transcribed sequences. In other embodiments, DNA sequences derived from the SV40 splice, and polyadenylation sites may be used to provide the non-transcribed genetic elements.

As described herein, in some embodiments, the present invention provides truncated PD-1 constructs for use in cellular based therapies. In some embodiments, T-cells or NK-cells are modified to comprise a) a PD-1 construct described herein; and b) an immune system targeting molecule. As used herein, the term “immune system targeting molecule” refers to any agent that renders a T-cell or NK-cell useful for therapeutic purposes. In some embodiments, the immune system targeting molecule targets the T-cell or NK-cell to a particular antigen (e.g., cancer or autoimmune disease antigen). In some embodiments, the immune system targeting molecule is a chimeric antigen receptor or a T-cell receptor. In some embodiments, NK-cells are induced to have T-cell like activity by recombinant expression of CD3-chains and a TCR to form a functional TCR-complex as described in WO2016116601.

Cancer immunotherapy is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates).

Active immunotherapy directs the immune system to attack tumor cells by targeting TAAs. Passive immunotherapies enhance existing anti-tumor responses and include the use of monoclonal antibodies, lymphocytes and cytokines.

Active cellular therapies usually involve the removal of immune cells from the blood or from a tumor. Those immune cells specific for the tumor are cultured and returned to the patient whit the purpose to attack the tumor. Cell types that can be used in this way are natural killer cells, lymphokine-activated killer cells, cytotoxic T-cells and dendritic cells. One US-approved cell-based therapy is Dendreon's Provenge, for the treatment of prostate cancer.

Adoptive T-cell therapy is a form of passive immunization by the transfusion of T-cells. T-cells are found in blood and tissue and usually become activate upon finding foreign pathogens. T-cells are specifically activated when the TCRexpressed on the cell surface encounters cells that display on their surface the cognate antigenic peptide that typically are derived from foreign proteins. These cells can be either infected cells, tumor cells or antigen presenting cells (APCs). T-cells are found in normal tissues and in tumor tissues, where they are known as tumor infiltrating lymphocytes (TILs). T-cells are activated by the presence of APCs such as dendritic cells that present tumor antigens. Although T-cells can attack the tumor, the environment within the tumor is highly immunosuppressive, preventing immune-mediated tumor death.

T-cell-based therapies have become increasingly attractive during the past decades. Technically two main methods have been exploited: 1) the isolation of patient's own T-cells derived from peripheral blood or tumor sites, known as Tumor Infiltrating Lymphocytes (TILs). These T-cells are expanded ex-vivo and re-injected in the patient. 2) TILs or any functional T-cell are isolated from a responding patient (i.e. vaccinated patient). The T-Cell Receptor (TCR) of these TILs is subsequently identified (DNA and protein sequences), cloned and expressed in T-cells from a HLA-matched patient, in order to redirect these T-cells against a tumor. In addition, other targeting molecules such as Chimeric Antigen Receptors (CARs), comprising an antigen binding domain linked to the signaling domain of the TCR, can also be used to redirect T-cell specificity against tumor.

T-cells may target any antigen (e.g., cancer antigen) including but not limited to proteins, subunits, domains, motifs, and/or epitopes belonging to the following list of target antigens, which includes both soluble factors such as cytokines and membrane-bound factors, including transmembrane receptors: 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CDlla, CDllb, CDllc, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAMS, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxinl, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta7, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpl, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Mucl), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEMS, TEM7, TEM8, TERT, testicular PLAP-like alkaline phosphatase, TfR, TGF, TGF-alpha, TGF-beta, TGF-beta Pan Specific, TGF-beta R1 (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R3Apo-2, DR4), TNFRSF10B (TRAIL R2DR5, KILLER, TRICK-2A, TRICK-B), TNFRSF10C (TRAIL R3DcR1, LIT, TRID), TNFRSF10D (TRAIL R4DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF R1CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3M68, TR6), TNFRSF7 (CD27), TNFRSF8 (CD30), TNFRSF9 (4-1 BB CD137, ILA), TNFRSF21 (DR6), TNFRSF22 (DcTRAIL R2TNFRH2), TNFRST23 (DcTRAIL R1 TNFRH1), TNFRSF25 (DR3Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-α Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (fit-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and receptors for hormones and growth factors.

One skilled in the art will appreciate that the aforementioned list of targets refers not only to specific proteins and biomolecules, but the biochemical pathway or pathways that comprise them. For example, reference to CTLA-4 as a target antigen implies that the ligands and receptors that make up the T-cell co-stimulatory pathway, including CTLA-4, B7-1, B7-2, CD28, and any other undiscovered ligands or receptors that bind these proteins, are also targets. Thus, target as used herein refers not only to a specific biomolecule, but the set of proteins that interact with said target and the members of the biochemical pathway to which said target belongs.

In some embodiments of the present invention, methods and compositions are provided for the treatment of tumors and immune system disorders. In some embodiments, the cancer is, for example, lung cancer, breast cancer, pancreatic cancer, prostate cancer, melanoma or multiple myeloma.

Other cell proliferative disorders, or cancers, contemplated to be treatable with the methods of the present invention include human sarcomas and carcinomas, including, but not limited to, fibrosarcoma, myxosarcoma, liposarcoma, chondrosarcoma, osteogenic sarcoma, chordoma, angiosarcoma, endotheliosarcoma, lymphangiosarcoma, Ewing's tumor, lymphangioendotheliosarcoma, synovioma, mesothelioma, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemias, acute lymphocytic leukemia and acute myelocytic leukemia (myeloblastic, promyelocytic, myelomonocytic, monocytic and erythroleukemia), chronic leukemia (chronic myelocytic (granulocytic) leukemia and chronic lymphocytic leukemia), polycythemia vera, lymphoma (Hodgkin's disease and non-Hodgkin's disease), multiple myeloma, Waldenstrom's macroglobulinemia, and heavy chain disease.

Tumor cell resistance to chemotherapeutic agents represents a major problem in clinical oncology. In some embodiments, compositions and methods of the present invention provides means of ameliorating this problem by effectively administering a combined therapy approach. However, it should be noted that traditional combination therapies may be employed in combination with the compositions of the present invention. For example, in some embodiments of the present invention, immunotherapies are used before, after, or in combination with the traditional therapies.

Alternatively, immunotherapy with the methods described herein precedes or follows the other agent treatment by intervals ranging from minutes to weeks. In embodiments where the other agent and immunotherapy are applied separately to the patient, one would generally ensure that a significant period of time did not expire between the time of each delivery, such that the fusion protein and chemotherapeutic agent would still be able to exert an advantageously combined effect on the patient. In such instances, it is contemplated that cells are contacted with both modalities within about 12-24 hours of each other and, more preferably, within about 6-12 hours of each other, with a delay time of only about 12 hours being most preferred. In some situations, it may be desirable to extend the time period for treatment significantly, however, where several days (2 to 7) to several weeks (1 to 8) lapse between the respective administrations.

In some embodiments of the invention, one or more methods or cells of the invention and an additional active agent are administered to a subject, more typically a human, in a sequence and within a time interval such that the compound can act together with the other agent to provide an enhanced benefit relative to the benefits obtained if they were administered otherwise. For example, the additional active agents can be co-administered by co-formulation, administered at the same time or administered sequentially in any order at different points in time; however, if not administered at the same time, they should be administered sufficiently close in time to provide the desired therapeutic or prophylactic effect. In some embodiments, the compound and the additional active agents exert their effects at overlapping time points. Each additional active agent can be administered separately, in any appropriate form and by any suitable route. In other embodiments, the compound is administered before, concurrently or after administration of the additional active agents.

In various examples, the cells and the additional active agents are administered less than about 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In other examples, the compound and the additional active agents are administered concurrently. In yet other examples, the cells and the additional active agents are administered concurrently by co-formulation.

In other examples, the cells and the additional active agents are administered at about 2 to 4 days apart, at about 4 to 6 days apart, at about 1 week part, at about 1 to 2 weeks apart, or more than 2 weeks apart.

In certain examples, the cells and optionally the additional active agents are cyclically administered to a subject. Cycling therapy involves the administration of a first agent for a period of time, followed by the administration of a second agent and/or third agent for a period of time and repeating this sequential administration.

Cycling therapy can provide a variety of benefits, e.g., reduce the development of resistance to one or more of the therapies, avoid or reduce the side effects of one or more of the therapies, and/or improve the efficacy of the treatment.

In other examples, one or more cells of some embodiments of the present invention and optionally the additional active agent are administered in a cycle of less than about 3 weeks, about once every two weeks, about once every 10 days or about once every week. One cycle can comprise the administration of an inventive T-cell and optionally the second active agent by infusion over about 90 minutes every cycle, about 1 hour every cycle, about 45 minutes every cycle, about 30 minutes every cycle or about 15 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

Courses of treatment can be administered concurrently to a subject, i.e., individual doses of the additional active agents are administered separately yet within a time interval such that the inventive compound can work together with the additional active agents. For example, one component can be administered once per week in combination with the other components that can be administered once every two weeks or once every three weeks. In other words, the dosing regimens are carried out concurrently even if the therapeutics are not administered simultaneously or during the same day.

In some embodiments of the present invention, the regional delivery of modified cells to patients with cancers is utilized to maximize the therapeutic effectiveness of the delivered agent. Similarly, the chemo- or radiotherapy may be directed to a particular, affected region of the subject's body. Alternatively, systemic delivery of the immunotherapeutic composition and/or the agent may be appropriate in certain circumstances, for example, where extensive metastasis has occurred.

In addition to combining the cells of some embodiments of the present invention with chemo- and radiotherapies, it also is contemplated that traditional gene therapies are used.

An attractive feature of the present invention is that the therapeutic compositions may be delivered to local sites in a patient by a medical device. Medical devices that are suitable for use in the present invention include known devices for the localized delivery of therapeutic agents. Such devices include, but are not limited to, catheters such as injection catheters, balloon catheters, double balloon catheters, microporous balloon catheters, channel balloon catheters, infusion catheters, perfusion catheters, etc., which are, for example, coated with the therapeutic agents or through which the agents are administered; needle injection devices such as hypodermic needles and needle injection catheters; needleless injection devices such as jet injectors; coated stents, bifurcated stents, vascular grafts, stent grafts, etc.; and coated vaso-occlusive devices such as wire coils.

Exemplary devices are described in U.S. Pat. Nos. 5,935,114; 5,908,413; 5,792,105; 5,693,014; 5,674,192; 5,876,445; 5,913,894; 5,868,719; 5,851,228; 5,843,089; 5,800,519; 5,800,508; 5,800,391; 5,354,308; 5,755,722; 5,733,303; 5,866,561; 5,857,998; 5,843,003; and 5,933,145; the entire contents of which are incorporated herein by reference. Exemplary stents that are commercially available and may be used in the present application include the RADIUS (SCIMED LIFE SYSTEMS, Inc.), the SYMPHONY (Boston Scientific Corporation), the Wallstent (Schneider Inc.), the PRECEDENT II (Boston Scientific Corporation) and the NIR (Medinol Inc.). Such devices are delivered to and/or implanted at target locations within the body by known techniques.

### EXAMPLES

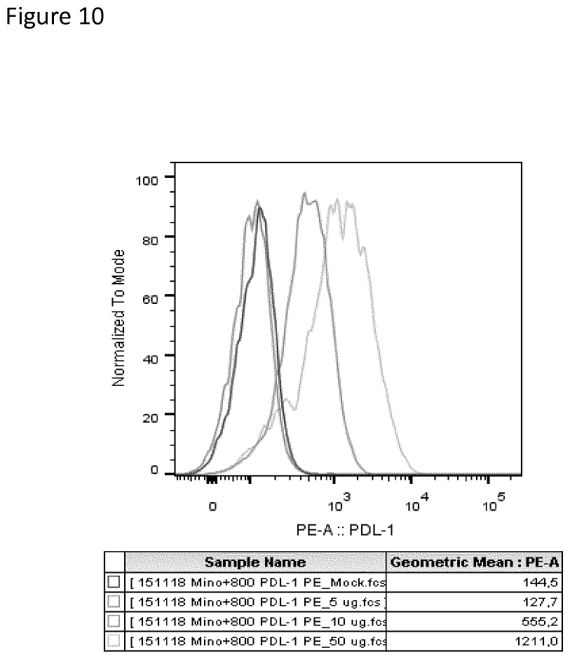

PDL-1/PD-1 cloning: the human gene encoding for PDL-1 FL (full-length) isoform 1 (long, http://www.uniprot.org/Q9NZQ7) was artificially synthesized (with a modification at the MfeI site) and subcloned into pENTR from Gateway system, Invitrogen. It was then recombined in mRNA synthesis vector (pCIpA102) or retroviral vector (pMP71) as in Walchli et al. 2011 The construct was then tested as mRNA in target cells (increasing amounts) and detected 12 hours later with specific antibody in flow cytometry (FIG. 10).

The PD-1 construct was ordered in Sino Biological Inc. as cDNA (pMD18-T-simple-PDCD1human PD-1 cDNA, HG10377-M) and amplified with specific primers

(PD-1f:
CACCATGCAGATCCCACAGGCGC,
PD-1r:
TTCTCGAGTCAGAGGGGCCAAGAGCAGTG,
PD-1trur:
TTCTCGAGTCAAGAGAACACAGGCACGGCTG)

to be subcloned into pENTR as a full length or a truncated gene. After sequence verification, it was recombined into pCIpA102 and pMP71 mRNA synthesis.

Effector cell electroporation was performed as follows:

1) 15/30 million/mL effector cells (expanded T-cells) were electroporated per each condition

2) Electroporation is performed in 4 mm gap cuvette, 0.5 ml RPMI, pulse 500V/2 ms

3) mRNA is used at about 0.1 ug/mL, if more than one type mRNA is transfected use 50% of each

Effector cells retroviral transduction was performed as follow:

Functional assays were performed as follows. For CD107a and CD107a/TNFalpha detections effector cells and target cells were electroporated with 0.1 μg/mL mRNA (description of mRNA) and left for 8-12 hours. Cells were mixed at a ratio (E:T=1:2) for 5-6 hours at 37° C. in the presence of Golgi plug and Golgi stop to prevent release of cytokines (TNFalpha) and CD107a. Used at recommended concentration.

Killing: the non-radioactive Europium TDA (EuTDA) cytotoxicity assay based on

PerkinElmer's DELFIA technology was performed. The EuTDA assay uses time-resolved fluorometry (TRF) and the measured fluorescence signal correlates directly with the amount of lysed cells.

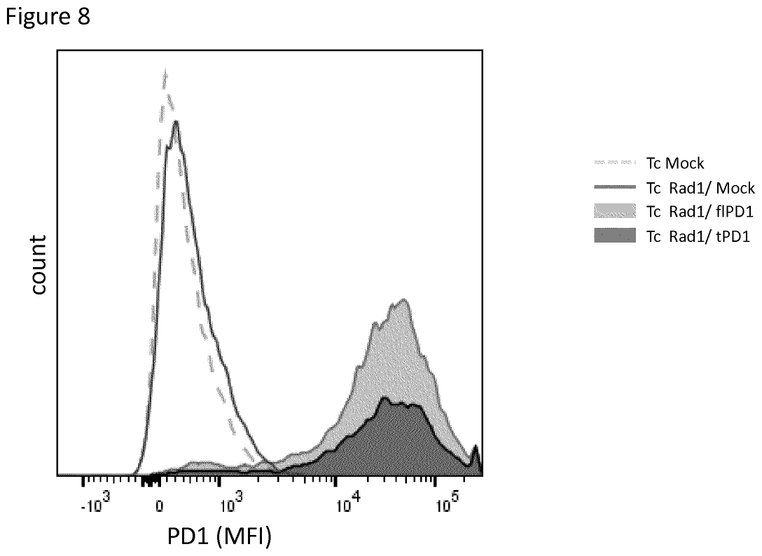

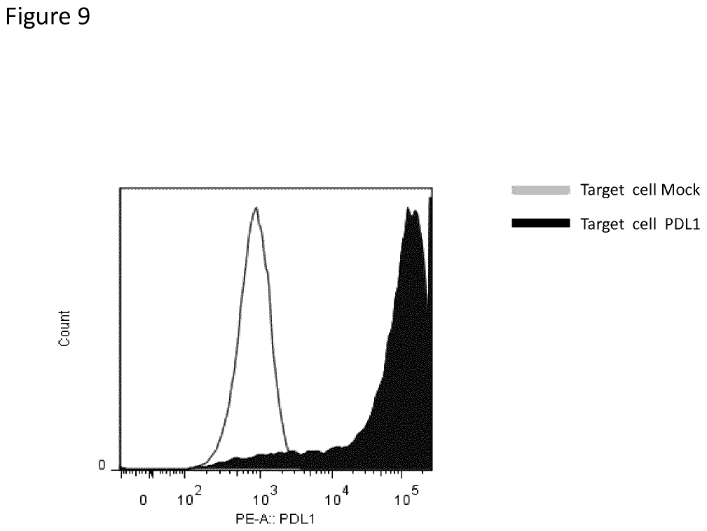

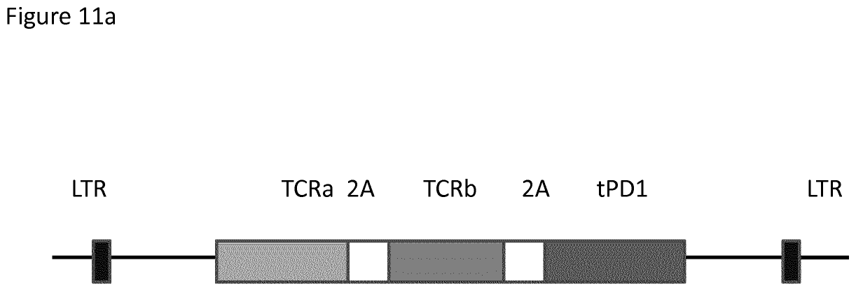

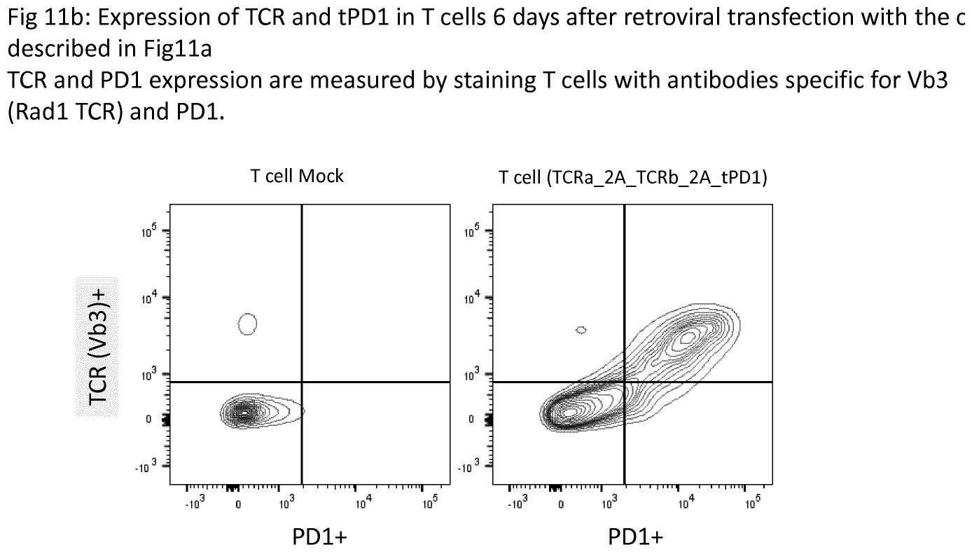

All the constructs are well expressed. FIG. 8 shows a representative staining of T-cells electroporated with mRNA encoding tPD-1 (SEQ ID NO:2) and flPD-1 (SEQ ID NO:1, left panel) and staining of Radium-1 TCR using a specific anti-Vb antibody (FIG. 11b). FIG. 9 is a representative staining of PDL-1 expression in K562 cells after electroporation.

### Example 1 Effector Function Marker of Natural Killer Cell Line

This experiment was constructed to compare a PD-1 molecule according to the invention, wherein the intracellular domain comprises a juxtamembrane domain and wherein the intracellular domain further is truncated such as it is lacking other signaling domains with a full-length PD-1 having normal suppression signaling.

Target cells were PDL-1 negative cells (EBV-LCL, K562 or K562-HLA-A2, (a derivative of K562 cells (ATCC® CCL-243™) stably transduced with a HLA-A2 construct), which have been or not electroporated with PDL-1 mRNA, in order to demonstrate that the observed improvement was a consequence of truncated PD-1 binding to PDL-1. As shown in FIG. 1, the tPD-1 construct (SEQ ID NO: 2) improves effector stimulation (increased expression of CD107a+, a degranulation marker shown to correlate with the killing abilities of effector cells) compared to not transduced or flPD-1 (SEQ ID NO:1) transduced NK-92 cells. The killing here is NK receptor dependent as NK-92 cell line is an NK derivative it will naturally recognize K562 due to their lack of HLA class I molecule at the surface (missing self).

The truncated PD-1 receptor may improve target recognition and the effector functions, such as degranulation, as indicated by increased expression of CD107a in NK-92 cells engineered to express the truncated PD-1 receptor (SEQ ID NO:2) when compared to either mock transfected NK-92 cells or NK-92 transfected to over-express the wild-type PD-1 receptor (SEQ ID NO:1). The engagement of the truncated PD-1 receptor with its ligand (PDL-1), expressed on target cells, seems to block the inhibitory check-point.

### Example 2 Cytotoxicity of Natural Killer Cell Line

Effector cells, in this case NK-92 cells (FIG. 2), were stimulated for 2 hours with target cells (K562 cells), either PDL-1 negative or genetically modified to express PDL-1 and labeled with BATDA at Effector to Target ratio (E:T) of 25:1. Measurement of the fluorescence signal was performed on a VICTOR™ plate reader and the data analyzed using GraphPad prism software (GraphPad Software, Inc., La Jolla, Calif., USA). NK-92 mock for PD-1, NK-92 transfected (electroporation) to express the full length PD-1 (SEQ ID NO:1) or the truncated PD-1 (SEQ ID NO:2) were tested in the assay. These results are complementing the ones presented in FIG. 1 and demonstrate an increased killing capacity of the NK-92 cells expressing tPD-1. In addition, they also show that if the target cells are PDL-1 negative, no effect (negative or positive) are observed on the target killing compared to mock or flPD-1 transfected. This is important because the effect of the truncated version in a PDL-1 free system could not be predicted, but according to US20160256488, the truncated PD-1 molecule therein seems to have a negative effect. Our construct does not induce this negative effect on the effector cells killing function and is neutral in a PDL-1 free system.

### Example 3 Effector Function Marker of Natural Killer Cell Line Expressing TCR-Complex

UK-92-TCR is a derivative of NK-92 cells (see WO2016116601), we herein test the effect of the tPD-1 construct when the TCR recognition is guiding the killing (FIG. 3a, 3b), we have used 2 different clinical TCRs in order to demonstrate that the effect was not TCR dependent: Radium-1 TCR (specific for TGFBRII antigen presented by HLA-A2, see WO2017194555). In the case of specific killing (+peptide), the target cell killing is improved for PDL-1 compared to no PDL-1, suggesting that tPD-1 is improving the killing and, in this case, increasing the target recognition. This is a demonstration that the extracellular domain of PD-1 is improving the recognition probably by reinforcing the binding to target cells. As expected flPD-1 is inhibiting the process only if PDL-1 is expressed. Taken together our data show that tPD-1 is improving UK-92-TCR in their killing ability.

### Example 4 Effector Function Markers of T-Cells

Further experiments demonstrated improved target recognition and effector functions with tPD-1 in T-cells (FIG. 4). We herein monitored the degranulation of the T-cells by analyzing the CD107a+ expression. T-cells were electroporated with mRNA to express either Radium-1 TCR (specific for TGFBRII antigen presented by HLA-A2, see WO2017194555) or Radium-1 TCR plus flPD-1 (SEQ ID NO:1) or Radium-1 TCR plus tPD-1 (SEQ ID NO:2). Mock T-cells were used as negative control. K562-A2 cells were used as target cells. K562-A2 cells were either mock or PDL-1 transfected and loaded with the cognate peptide (TGFBRII, p621) or an irrelevant one, namely MARTI pep (as control). T-cell specific activation was measured after 6 h of co-culture by flow cytometry-based assay as CD107a expression (in duplicates). The data confirm the results obtained in NK-92 cells; over-expression of flPD-1 receptor (SEQ ID NO:1) reduces/blocks T-cell activation when target cells express PDL-1 ligand while over-expression of the tPD-1 receptor (SEQ ID NO:2) restores T-cell activation when target cells express PDL-1 ligand. The T-cell response is stronger (Radium-1 TCR/tPD-1 versus Radium-1 TCR). We next repeated the experiment and monitored the cytokine release which is an important feature of effector T-cells. tPD-1 could well inhibit normal effector function as the juxtamembrane domain function is not properly described but might well have regularity function that could potentially affect TCR-stimulation outcome. As shown the TNFalpha release in both CD4 (FIG. 5a) or CD8 (FIG. 5b) followed CD107a (FIG. 4), meaning that tPD-1 improved the cytokine release when target cells expressed PDL-1. This is supporting the positive effect of tPD-1 on general effector function depending on TCR in T-cells.

### Example 5 Cytotoxicity of TcmRNA Electroporated

Measurement of killing function was also performed by BLI technology (see FIG. 6).

The non-radioactive bioluminescence (BLI)-based cytotoxicity assay was performed to measure the cytotoxic activity of effector cells overtime. BLI assay is based on the detection of light from the enzyme luciferase by using luminometers. Emission of bioluminescence in luciferase-expressing cells decreases when cells are dying.

Effector cells (T-cells) were co-cultured with luciferase-transduced target cells HCT 116 cells (HLA-A2 restricted/TGFBRII frameshift) and RV infected to express PDL-1 ligand and incubated at Effector to Target ratio (E:T) 25:1. Bioluminescence emission was measured over time, at different time points on a VICTOR X4™ plate reader and cytolytic activity of T-cells was measured.

Briefly, in FIG. 5, T-cells expressing the therapeutic TCR (Radium-1); T-cells expressing both the therapeutic TCR (Radium-1) and the full length PD-1 (SEQ ID NO:1), T-cells expressing both the therapeutic TCR (Radium-1) and the truncated PD-1 (SEQ ID NO:2) were incubated with the tumor cell line Granta HLA-A2 that were either mock or PDL-1 transfected and O/N loaded with the indicated peptide at 10 μM. The kinetic in killing is improved in the presence of tPD-1 when target cells are PDL-1 positive, probably due to i) the exclusion of endogenous PD-1 from the immune synapse, thus inhibiting the negative signal and ii) the increase in avidity of the effector cell for the target cell through tPD-1 binding to PDL-1.

### Example 6 Cytotoxicity of T-Cells Retrovirally Infected

T-cells were transduced with the construct designed as in FIG. 11, containing Radium-1 TCR fused to either tPD-1 or flPD-1. Expression is shown in F11.

Briefly, in FIG. 7, T-cells expressing the therapeutic TCR (Radium-1); T-cells expressing both the therapeutic TCR (Radium-1) and the full length PD-1 (SEQ ID NO:1) and T-cells expressing both the therapeutic TCR (Radium-1) and the truncated PD-1 (SEQ ID NO:2) were incubated with the tumor cell line Granta HLA-A2 that were either mock or PDL-1 transfected and O/N loaded with the indicated peptide at 10 μM.

The Radium-1 TCR fused to either tPD-1 or flPD-1 constructs were compared with the TCR alone and the % of lysis is plotted against the log of the time. As shown the tPD-1 kinetic is improved and plateau is reached fast, whereas the flPD-1 is inhibiting the killing, the TCR only control is situated mid-way and shows the kinetics of a TCR with a good affinity. Therefore, although Radium-1 is an efficient TCR (see WO2017194555), its kinetic can still be improved by tPD-1. Consequently tPD-1 expressing T-cells may kill their target faster. In addition, it shows that even if tPD-1 binds to PDL-1 and enforce the immune synapse, it doesn't block the effector cell on its target, it rather seems to improve the serial killing.

### Example 7 Construct Expression mRNA Electroporation

mRNA encoding tPD-1 and flPD-1 were electroporated in PBMC derived T-cells and stained using an antibody targeted against the extracellular domain of PD-1 (FIG. 8). As shown, the intensity of the tPD-1 construct is not different of the one of flPD-1 which suggests that the plasma membrane targeting is not affected by the truncation, thus tPD-1 is not retained in intracellular compartment, or simply degraded. Protein degradation could be a major issue when signaling domains are removed, and the design of our construct does not seem to affect the stability of the protein. In addition, the fact that the antibody is recognizing both constructs as efficiently demonstrates that tPD-1 is correctly folded on its extracellular part, thus the juxtamembrane domain excluding the ITIM does not affect correct folding and targeting of tPD-1 in the effector cells.

The PDL-1 construct that we have used for our experiments is also well produced and expressed (FIG. 9).

### Example 8 Design and Expression of the Retroviral Construct

We propose to use a viral construct for the expression of the therapeutic TCR together with tPD-1. We take advantage of the 2A skipping sequence in order to obtain equimolar amounts of both proteins (TCR and tPD-1). T-cells were transduced and stained for TCR expression (Vb3 antibody) and PD-1 expression. As shown, the construct is efficient in producing both proteins in an equimolar way.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. A PD-1 molecule comprising an extracellular domain, a transmembrane domain and an intracellular domain, wherein the intracellular domain comprises a juxtamembrane domain and wherein the intracellular domain is lacking other signaling domains.

2. The PD-1 molecule according to claim 1, wherein the juxtamembrane domain comprises an amino acid sequence corresponding to SEQ ID NO 8 (CSRAARGTIGARRTGQPLKEDPSAVPVFS).

3. The PD-1 molecule according to claim 1, wherein the intracellular domain consists of an amino acid sequence corresponding to SEQ ID NO 8 (CSRAARGTIGARRTGQPLKEDPSAVPVFS).

4. The PD-1 molecule according to claim 1, wherein the juxtamembrane domain consists of an amino acid sequence corresponding to SEQ ID NO 9 (ICSRAARGTIGARRTGQPLKEDPSAVPVFS).

5. An immune effector cell expressing the PD-1 molecule according to claim 1 in its cell membrane.

6. The immune effector cell according to claim 5, further comprising a receptor with affinity for a tumor associated antigen in its cell membrane.

7. The immune effector cell according to claim 5, wherein the PD-1 molecule comprises an amino acid sequence corresponding to SEQ ID NO:3 or sequences that are at least 97% identical to SEQ ID NO:3.

8. The immune effector cell according to claim 5, wherein the PD-1 molecule comprises the amino acid sequence SEQ ID NO:4 or sequences that are at least 97% identical to SEQ ID NO:4 provided that the underlined position comprises a leucine residue.

9. The immune effector cell according to claim 5, wherein the immune effector cell is selected from the group consisting of cytotoxic T-cells, T-helper cells and natural killer cells.

10. The immune effector cell according to claim 5, wherein the immune effector cell is a natural killer cell line.

11. A method comprising, administering the immune effector cell according to claim 5 to a patient in need of immunotherapy or to generate an immune response in the patient.

12. The method according to claim 11, wherein immune effector cell is administered to generate a tumor antigen-specific immune response.

13. The method according to claim 12, wherein the immune effector cell is a T-cell or NK cell expressing a high affinity truncated PD-1 comprising the extracellular domain, transmembrane domain, and juxtamembrane domain and lacking the signaling domain.

14. A method of generating an antigen-specific immune response in a patient, comprising:
administering to the patient a T-cell or NK cell expressing 1) a high affinity truncated PD-1 comprising the extracellular domain, transmembrane domain, and juxtamembrane domain and lacking the signaling domain and 2) an immune system targeting molecule.

15. The method of claim 14, wherein said immune system targeting molecule is a T-cell receptor.

16. The method of claim 14, wherein said PD-1 comprises an amino acid sequence selected from the group consisting of SEQ ID NO:2 and sequences that are at least 90% identical to SEQ ID NO:2.

17. The method of claim 14, wherein position 132 in said PD-1 comprises a Leucine residue.

18. The method of claim 14, wherein said antigen is a cancer antigen.

19. A circular or isolated nucleic acid encoding the PD-1 molecule according to claim 1.

20. The circular or isolated nucleic acid according to claim 19, encoding a protein as defined by SEQ ID NO:2 or sequences that are at least 97% identical to SEQ ID NO:2.

21. The circular or isolated nucleic acid according to claim 19, further comprising a sequence encoding a TCR.

22. The circular or isolated nucleic acid according to claim 21, comprising a ribosome skipping sequence between the sequence encoding the PD-1 molecule and the sequence(s) encoding the TCR chains.


SEQUENCE LISTING (12 sequences)

SEQ ID NO: 1 - protein, 288 aa, homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 220 aa, homo sapiens
  MISC_FEATURE (132)..(132): X is A, L, I or V
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKXQIKESLRAELRVTERRAEVPTAHPSPSPRPAGQFQTLVVGVVGGLLGSLVLLVWVLAVICSRAARGTIGARRTGQPLKEDPSAVPVFS

SEQ ID NO: 3 - protein, 200 aa, homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFS

SEQ ID NO: 4 - protein, 200 aa, homo sapiens
  MISC_FEATURE (112)..(112): X is L, I or V
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKXQIKESLRAELRVTERRAEVPTAHPSPSPRPAGQFQTLVVGVVGGLLGSLVLLVWVLAVICSRAARGTIGARRTGQPLKEDPSAVPVFS

SEQ ID NO: 5 - protein, 6 aa, homo sapiens
VDYGEL

SEQ ID NO: 6 - protein, 6 aa, homo sapiens
TEYATI

SEQ ID NO: 7 - protein, 23 aa, homo sapiens
AARGTIGARRTGQPLKEDPSAVP

SEQ ID NO: 8 - protein, 29 aa, homo sapiens
CSRAARGTIGARRTGQPLKEDPSAVPVFS

SEQ ID NO: 9 - protein, 30 aa, homo sapiens
ICSRAARGTIGARRTGQPLKEDPSAVPVFS

SEQ ID NO: 10 - DNA, 23 bp, homo sapiens
caccatgcagatcccacaggcgc

SEQ ID NO: 11 - DNA, 29 bp, homo sapiens
ttctcgagtcagaggggccaagagcagtg

SEQ ID NO: 12 - DNA, 31 bp, homo sapiens
ttctcgagtcaagagaacacaggcacggctg